(19) United States
(12) Patent Application Publication
(10) Pub. No.: US 2025/0195770 A1
(43) Pub. Date: Jun. 19, 2025

(54) RADIATION-PROTECTION DEVICE FOR A SYRINGE

(71) Applicant: LEMER PAX, La Chapelle Sur Erdre (FR)

(72) Inventor: Pierre-Marie LEMER, NANTES (FR)

(21) Appl. No.: 18/849,832

(22) PCT Filed: Mar. 23, 2023
(86) PCT No.: PCT/EP2023/057473

§ 371 Date: Sep. 23, 2024

(30) Foreign Application Priority Data
Mar. 24, 2022 (FR) FR2202620

(51) Int. Cl. A61M 5/178

(52) U.S. Cl. CPC A61M 5/1785

(57) ABSTRACT

The present invention relates to a radiation-protection device (1) intended to be fitted onto a syringe used for injecting a radioactive product. This radiation-protection device (1) comprises a tubular radiation-protection structure (11) equipped with a lateral housing (118) to accommodate a shield (12) made from a transparent radiation-protection material. One of the ends (121) of the shield (12) made of transparent radiation-protection material comprises a tenon structure (14a) designed to engage with a complementary mortise structure (14b) formed in said lateral housing (118); and a retractable locking member (14c) is designed to engage with another end (122) of said shield (12) made of transparent radiation-protection material, in order to lock said shield in position.

### BACKGROUND OF THE INVENTION

The present invention relates to the technical field of the radiation-protection devices of the “syringe-shield” type, which are intended to be fitted onto the syringes used for injecting radioactive product(s), to protect operators from ionising radiation.

### PRIOR ART

Some sectors of activity require the handling of radioactive products that emit ionising radiation, such as electromagnetic radiation (X, gamma) and/or particulate radiation (alpha, beta, neutrons).

In the specific field of nuclear medicine, radioactive products are used to implement diagnostic and/or treatment techniques, such as in vivo functional imaging (e.g. scintigraphy), in vitro biological diagnostics (in particular radioimmunology) and metabolic radiotherapy.

These radioactive products are often administered to the patient using a syringe onto which is added a radiation-protection device capable of attenuating ionising radiation, in particular to protect the operator handling the syringe.

Such radiation-protection devices, commonly called “syringe-shields”, are for example described in documents EP-1 317 299, or FR-2 971 425.

This type of device generally comprises a tubular radiation-protection envelop that includes:—an inner surface, intended to cover at least part of the outer surface of the cylindrical body of the syringe, hereinafter called “syringe body”, and—an outer surface, intended to be held by the operator.

This tubular envelop comprises—a front opening, through which the front end of the syringe body provided with a liquid suction and ejection hole is intended to emerge,—a rear opening, for inserting and extracting said syringe body, and for operating the plunger thereof, and often—a transparent shield made of a radiation-protection material, for visual access to the outer surface of the syringe body (in such a way as to visualize the level of the front end of the plunger inside the syringe body).

However, in the event of breakage or damage, the transparent shields made of radiation-protection material used in current syringe-shield structures are not easy to replace. Moreover, these known syringe-shield structures are often complex and expensive.

### SUMMARY OF THE INVENTION

In order to remedy the above-mentioned drawbacks of the state of the art, the present invention proposes a radiation-protection device intended (or designed) to be fitted onto a syringe used for injecting a radioactive product, said syringe comprising a cylindrical syringe body having a front end provided with a liquid suction and ejection hole and a rear end provided with an opening into which is inserted a syringe plunger, said radiation-protection device comprising:
(i) a tubular radiation-protection structure, comprising a longitudinal axis, said tubular radiation-protection structure being made at least partially of a radiation-protection material and comprising—an inner surface delimiting a central housing adapted to receive at least part of said syringe body,—an outer surface,—a front opening through which said front end of said syringe body is intended to emerge,—a rear opening adapted for inserting and extracting said syringe body, and for operating said syringe plunger,—a side opening that opens into said central housing, said side opening comprising a rebate structure and forming a side housing, and
   (ii) a shield made of a transparent radiation-protection material, added into said side housing and positioned in rest on said rebate structure,
said radiation-protection device also comprising locking means to lock said shield of transparent radiation-protection material in said side housing,
this radiation-protection device being characterized in that said locking means comprise
   a tenon structure arranged at one end of said shield of transparent radiation—protection material, adapted to cooperate with a complementary mortise structure arranged in said side housing, and
   a retractable locking member adapted (or designed) to cooperate with another end of said shield of transparent radiation-protection material, said locking member being able to be activated to lock said shield of transparent radiation-protection material in position after introduction of said tenon structure into said mortise structure, and to be deactivated to allow said shield of radiation-protection material and said tubular structure to be separated from each other.

Such a structure, very simple, makes it easy to fit and remove the shield of transparent radio-protective material.

Other non-limiting and advantageous features of the radiation-protection device according to the invention, taken individually or according to all the technically possible combinations, are the following:
the end of the shield of transparent radiation-protection material cooperating with the locking member includes a shield rebate for receiving said locking member, said shield rebate including a rebate bottom and a rebate edge, a bottom face of said locking member being intended to bear against said rebate bottom of said shield rebate, to axially lock said shield of radio-protective material, and a thickness of said locking member being intended to come between said rebate edge of said shield rebate and an end face opposite said side housing, to longitudinally lock said shield of radio-protective material;
the side housing of the radiation-protection device is elongated in shape and comprises a major axis that extends parallel to said longitudinal axis of said tubular radiation-protection structure, said side housing comprising an upstream end directed towards the rear opening of said tubular radiation-protection structure and a downstream end directed towards the front end of said tubular radiation-protection structure; said mortise structure is arranged at said downstream end of the lateral housing; said tenon structure is arranged at one end of said shield of transparent radiation-protection material intended to be oriented towards said downstream end of the lateral housing; and said retractable locking member is intended to cooperate with the end of said shield of transparent radiation-protection material positioned on the side of the upstream end of said lateral housing;
the retractable locking member consists of an elastic locking ring adapted to surround the tubular radiation-protection structure and the shield of transparent radiation-protection material;
the retractable locking member in the form of an elastic locking ring is made of rubber;
the retractable locking member in the form of an elastic locking ring comprises a flat structure that projects from the outer surface of said tubular radiation-protection structure, said projecting flat structure being adapted to serve as a bearing structure, to be stably put on a receiving plane;
the flat structure is diametrically opposed to the bottom face of the locking member;
the outer surface of the tubular radiation-protection structure includes a slot for the integration, at least partial, of said retractable locking member in the form of an elastic ring;
the outer surface of the tubular radiation-protection structure comprises at least a part of non-circular cross-section adapted to avoid or limit its rolling on a receiving surface.

Obviously, the different features, alternatives and embodiments of the invention can be associated with each other according to various combinations, insofar as they are not mutually incompatible or exclusive.

### DETAILED DESCRIPTION OF THE INVENTION

Moreover, various other features of the invention emerge from the appended description made with reference to the drawings that illustrate non-limiting embodiments of the invention, and wherein:

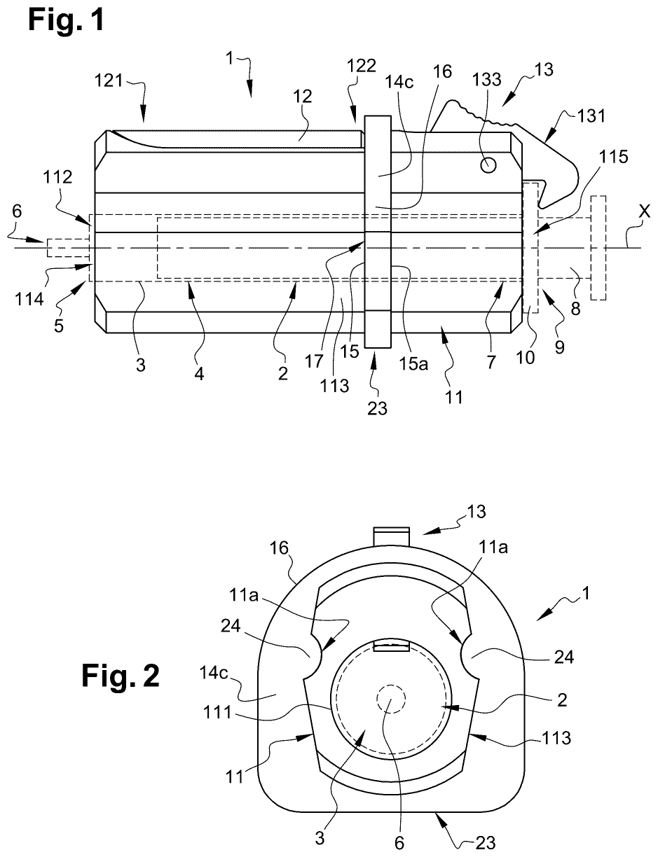

FIG. 1 is a side view of a first embodiment of a radiation-protection device according to the invention;

FIG. 2 is a front view of the radiation-protection device illustrated in FIG. 1;

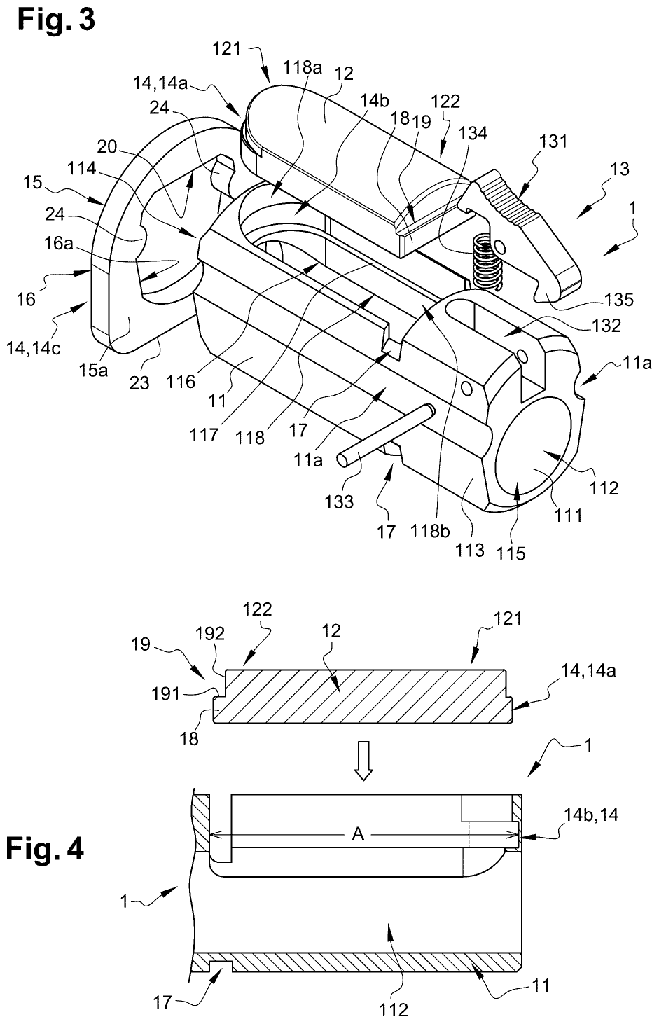

FIG. 3 is an exploded perspective view of the elements constituting the radiation-protection device illustrated in FIGS. 1 and 2;

FIG. 4 is a schematic view, in partial longitudinal cross-section, of the radiation-protection device illustrated in FIGS. 1 to 3, which shows an initial step of the operation of fitting the shield of transparent radiation-protection material onto the tubular radiation-protection structure;

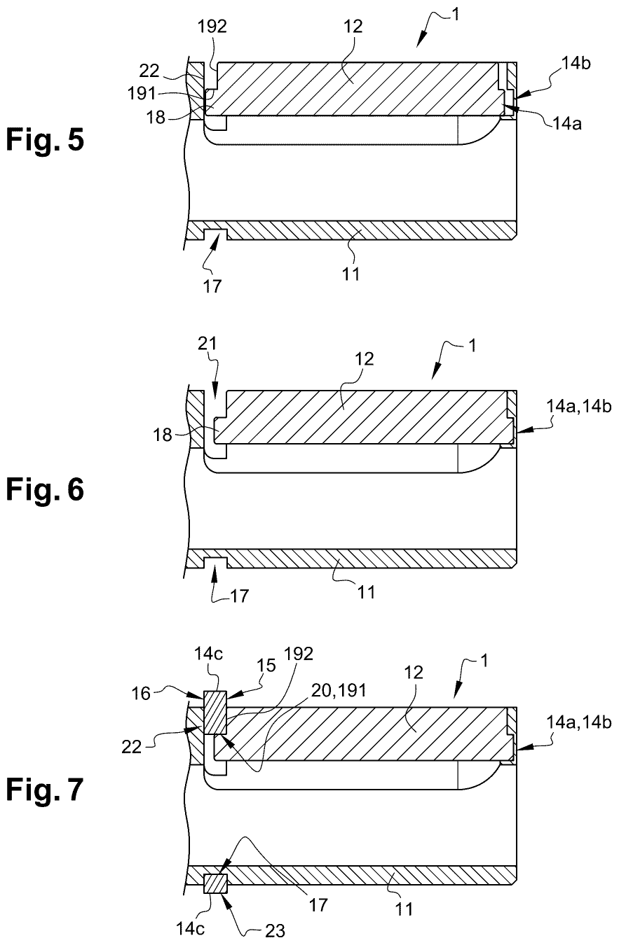

FIG. 5 is a view similar to FIG. 4, which shows a subsequent step of the operation of fitting the shield of transparent radiation-protection material onto the tubular radiation-protection structure;

FIG. 6 is a view similar to FIG. 5, which shows another subsequent step of the operation of fitting the shield of transparent radiation-protection material onto the tubular radiation-protection structure;

FIG. 7 is a view similar to FIG. 6, which shows still another subsequent step of the operation of fitting the shield of transparent radiation-protection material onto the tubular radiation-protection structure;

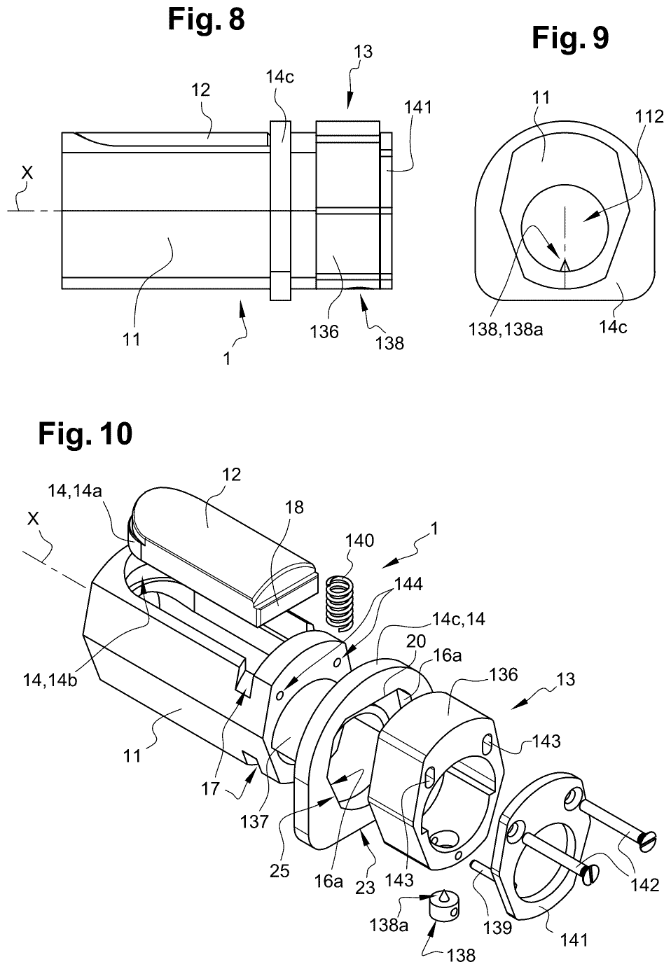

FIG. 8 is a side view of a second embodiment of a radiation-protection device according to the invention;

FIG. 9 is a front view of the radiation-protection device illustrated in FIG. 8;

FIG. 10 is an exploded perspective view of the elements constituting the radiation-protection device illustrated in FIGS. 8 and 9.

It is to be noted that, in these figures, the structural and/or functional elements common to the different alternatives can have the same references numbers.

The radiation-protection device 1 illustrated in FIGS. 1 to 3 is intended to be fitted onto a syringe 2 used for injecting a radioactive product.

The syringe 2, of conventional type, is illustrated in dotted lines in FIGS. 1 and 2; it comprises a cylindrical syringe body 3 having—an annular outer surface 4,—a front end 5, provided with a liquid suction and ejection hole 6, and—a rear end 7, at which is added a syringe plunger 8.

The rear end 7 of the cylindrical body 3 comprises a rear opening 9 for the passage of the syringe plunger 8; and this rear opening 9 is provided with a projecting rear flange 10.

The radiation-protection device 1, also called “syringe-shield”, comprises a tubular radiation-protection structure 11 and a shield 12 made of a transparent radiation-protection material, which, once associated together, are adapted to receive the syringe body 3 of the syringe 2.

This radiation-protection device 1 has for function to attenuate the ionising radiation emitted by the radioactive liquid sucked into the syringe body 3, and it thus protects the operators (as well as the patients) from the ionising radiation emitted by this liquid.

The radiation-protection device 1 also comprises locking means 13 adapted to removably (or releasably) lock the syringe 2 inside the tubular radiation-protection structure 11 associated with the shield 12 of transparent radiation-protection material.

The tubular radiation-protection structure 11 is in the general shape of a cylindrical body, of longitudinal axis X, made at least partially of a radiation-protection material, for example tungsten, lead, tantalum and more generally any material capable of attenuating ionising radiation.

This tubular radiation-protection structure 11 comprises:
an inner surface 111 delimiting a cylindrical central housing 112, adapted to receive at least part of the syringe body 3,
an outer surface 113,
a front opening 114, through which the front end 5 of the syringe body 3 is intended to emerge,
a rear opening 115, for inserting and extracting the syringe body 3, and for operating the syringe plunger 8,
a side opening 116 that opens into the central housing 112.

The cylindrical central housing 112 of the tubular radiation-protection structure 11 has a diameter that corresponds to, within the clearance, to the diameter of the annular outer surface 4 of the syringe body 3. The length of this central housing 112 corresponds approximately to the length of the syringe body 3.

The side opening 116 comprises a rebate structure 117 and forms generally a side housing 118 for receiving the shield 12 of transparent radiation-protection material. The rebate structure 117 extends over the whole or almost the whole periphery of the side opening 116, and it forms an annular bearing or seat surface for the shield 12 of transparent radiation-protection material.

The radiation-protection device 1 also comprises locking means 14 suitable to removably lock the shield 12 of transparent radiation-protection material in its side receiving housing 118, in rest against the rebate structure 117.

These locking means 14 comprise
a tenon structure 14a arranged at one end 121 of the shield 12 of transparent radiation-protection material,
a mortise structure 14b, complementary of the tenon structure 14a, and arranged in the side housing 118, and
a retractable locking member 14c adapted to cooperate with another end 122 of the shield 12 of transparent radiation-protection material.

The tenon structure 14a is made integral with the shield 12 of transparent radiation-

protection material.

The retractable locking member 14c here consists of an elastic locking ring adapted to surround the tubular radiation-protection structure 11 and the associated shield 12 of transparent radiation-protection material.

The elastic locking ring 14c is in the form of a flat ring of constant thickness delimited by two parallel flat faces 15 and 15a, an outer edge 16 and an inner edge 16a. This elastic locking ring 14c can be made of any material having elasticity characteristics. Preferably, it is made of rubber.

The tenon structure 14a of the shield 12 of transparent radiation-protection material is adapted to be introduced into the mortise structure 14b of the side housing 118. And the locking member 14c is adapted to be positioned (or activated) to lock the shield 12 of transparent radiation-protection material in position after introduction of the tenon structure 14a into said mortise structure 14b, and adapted to be deactivated (removed) to allow the shield 12 of radiation-protection material and the tubular radiation-protection structure 11 to be separated from each other.

In the embodiment illustrated, the side housing 118 is of elongated shape and has a major axis A (FIG. 4) that extends parallel to the longitudinal axis X of the tubular radiation-protection structure 11.

The mortise structure 14b is arranged at the downstream end 118a of the side housing 118 (directed towards the front opening 114 of the tubular radiation-protection structure 11). The tenon structure 14a is arranged at the downstream end 121 of the shield 12 of transparent radiation-protection material that is intended to be directed towards the downstream end 118a of the side housing 118. And the retractable locking member 14c is intended to cooperate with the front end 122 of the shield 12 of transparent radiation-protection material that is positioned on the side of the upstream end 118b of the side housing 118 (directed towards the rear opening 115 of the tubular radiation-protection structure 11).

The side housing 118 is over-sized in length, in the direction of its major axis A, to allow the engagement of the tenon structure 14a into the mortise structure 14b, as well as the operation of removing the tenon structure 14a out of the mortise structure 14b. The locking member 14c is adapted to surround the tubular radiation-protection structure 11/shield 12 of transparent radiation-protection material unit at the upstream end 122 of this shield 12 of transparent radiation-protection material.

The outer surface 113 of the tubular radiation-protection structure 11 includes a fitting slot 17 for the partial integration of the locking member 14c in the form of an elastic ring. This fitting slot 17 is arranged in a plane perpendicular to the longitudinal axis X of the tubular radiation-protection structure 11; it ensures a correct and stable positioning of the locking member 14c over the length of the tubular radiation-protection structure 11 and more particularly at the upstream end 122 of the shield 12 of transparent radiation-protection material.

As can be seen in particular in FIGS. 1 and 3 to 7, the upstream end 122 of the shield 12 of transparent radiation-protection material comprises a heel 18 that forms a shield rebate 19 delimited by a rebate bottom 191 and a rebate edge 192.

Once the shield 12 of transparent radiation-protection material positioned inside its side housing 118, the shield rebate 19 is directed outwards and, when the locking member 14c is in active position, a bottom face 20 of this locking member 14c comes in rest against the rebate bottom 191 of the shield rebate 19, to axially lock the shield 12 of radiation-protection material.

Moreover, the shield rebate 19 defines a groove 21 with the end face 22 opposite the side housing 118. And this groove 21 receives a thickness of the locking member 14c to longitudinally lock the shield 12 of radiation-protection material (in the direction of the longitudinal axis X of the tubular radiation-protection structure 11, and in the direction of the major axis A of the side housing 118). This thickness of the locking member 14c comes in position between the rebate edge 192 of the shield rebate 19 and the end face 22 opposite the side housing 118.

The groove 21 into which comes the locking member 14c is located in the continuation of the fitting slot 17 of the tubular radiation-protection structure 11.

The width of the groove 21, defined by the space separating the rebate edge 192 of the shield rebate 19 and the end face 22 of the side housing 118, corresponds to the thickness at this level of the locking member 14c, i.e. at the distance separating the two parallel flat faces 15 and 15a.

The structure and sizes of the different elements 11, 12 and 14c are adapted in such a way that, once the locking member 14c correctly positioned about the tubular radiation-protection structure 11 and the shield 12 of radiation-protection material, within the groove 21, this locking member 14c undergoes a minimum of stress (and even preferably no or practically no stress), in both tension and compression, to limit the degradations thereof in time and to optimize its lifetime.

As can be seen in FIGS. 1, 2 and 7 in particular, the locking member 14c in the form of an elastic ring is here simply partially fitted into the fitting slot 17 and into the groove 21. That way, its outer edge 16 projects at least in certain areas of the periphery of the tubular radiation-protection structure 11 and of the shield 12 radiation-protection material; and this makes it possible, in particular, to have peripheral gripping parts to facilitate the fitting and removal of the locking member 14c.

In an alternative embodiment, the locking member 14c can be totally integrated into the fitting slot 17 and into the groove 21.

The retractable locking member 14c here comprises a flat structure 23 on part of its outer edge 16, which projects from the outer surface 113 of the tubular radiation-protection structure 11. This flat structure 23 is adapted to serve as a bearing structure, so as to be stably put on a receiving plane. Preferably, this flat structure 23 is diametrically opposed to the bottom face 20 of the locking member 14c.

Starting from the tubular radiation-protection structure 11 dissociated from the shield 12 of radiation-protection material, as illustrated in FIG. 4, the shield 12 of radiation-protection material is introduced into the side housing 118 of the tubular radiation-protection structure 11 (FIG. 5), so as to come in rest on the rebated structure 117, and so that its tenon structure 14a is positioned facing the mortise structure 14b.

Then, the tenon structure 14a of the shield 12 of radiation-protection material is introduced into the mortise structure 14b of the tubular radiation-protection structure 11 (FIG. 6).

Finally, the locking member 14c is positioned around the tubular radiation-protection structure 11 and the shield 12 of radiation-protection material, within the fitting slot 17 and the groove 21, as illustrated in FIG. 7, this activation of the locking member 14c being possible due to the elasticity of its constitutive material.

In FIGS. 2 and 3 in particular, it can be noticed that the outer surface 113 of the tubular radiation-protection structure 11 comprises two longitudinal slots 11a, that extend over its whole length, parallel to the longitudinal axis X. These two longitudinal grooves 11a are adapted to receive protuberances 24, of complementary shape, formed on the inner edge 16a of the locking member 14c. These couples of slots 11a and protuberances 24 make it possible to ensure a correct angular positioning of the locking member 14c over the periphery of the tubular radiation-protection structure 11.

In an alternative embodiment, the mortise structure 14b can be provided on the side of the upstream end 118b of the side housing 118, the locking means 14 being then provided on the side of its downstream end 118a.

The locking means 13 for the syringe 2 are provided at the rear opening 115 of the tubular radiation-protection structure 11.

These locking means 13 here consist of a pivoting latch 131 mounted in a housing 132 on a pivot axis 133. One of the ends of the pivoting latch 131 is subjected to the action of a return spring 134, and its other end comprises a hook 135 suitable to block the projecting rear flange 10 of the rear end 7 of the syringe body 3, as illustrated in FIG. 1.

The return spring 134 is arranged to tend to place the hook 135 in active blocking position. The release of the syringe 2 is made by a manual action on the pivoting latch 131 against the force of the return spring 134.

FIGS. 8 to 10 illustrate a second embodiment of a radiation-protection device 1 according to the invention.

In this embodiment, the tubular radiation-protection structure 11 and the shield 12 of radiation-protection material are also present. The locking means 14 are also provided between the shield 12 of radiation-protection material and the tubular radiation-protection structure 11 (with the tenon structure 14a cooperating with the mortise structure 14b and the locking member 14c in the form of an elastic locking ring).

Here, the inner edge 16a of the elastic locking ring 14c still has the bottom face 20, but the protuberances 24 of the previous embodiment are replaced by cut-off corners 25.

This embodiment of FIGS. 8 to 10 essentially differs from the embodiment of FIGS. 1 to 3 in the structure of the locking means 13 for the syringe 2.

These locking means 13 are here made of an operating ring 136 mounted on a seat 137 with a possibility of movement perpendicular to the longitudinal axis X. The operating ring 136 is provided with a locking needle 138, the tip 138a of which is adapted to cooperate with the syringe body 3 (not shown in FIGS. 8 to 10).

The locking needle 138 is attached to the operating ring 136 by means of a pin 139. In active position, its tip 138a slightly projects (by a few tenth of millimetres) into the central housing 112 of the tubular radiation-protection structure 11 (FIG. 9).

Moreover, a return spring 140 is interposed between the seat 137 and an inner face of the operating ring 136 to tend to push the locking needle 138 against the syringe body 3 in place inside the central housing 112.

A blocking ring 141 ensures the holding in position of the operating ring 136. This blocking ring 141 is positioned at the end of the radiation-protection device 1 and is fixed to the tubular radiation-protection structure 11 by means of fixing screws 142 that pass through the operating ring 136 via oblong holes 143 and come into holes 144.

The fixing screw 142 are adapted so as not to clamp the manoeuvring ring 136 between the locking ring 141 and the tubular radiation protection structure 11, so as to allow movement of said manoeuvring ring 136 perpendicular to the longitudinal axis X. This movement is guided and limited by the fixing screws 142 associated with the oblong holes 143.

The positioning of a syringe 2 within the radiation-protection device 1 of FIGS. 8 to 10 is achieved by exerting a thrust on the operating ring 136 against the return force of the return spring 140, so as to retract the tip 138a of the locking needle 138 out of the central housing 112 of the tubular radiation-protection structure 11.

The syringe 2 can then be suitably positioned in the radiation-protection device 1. And the release of the operating ring 136 ensures the return to the active position of the tip 138a of the locking needle 138 (under the action of the return spring 140), against the cylindrical body 3 of the syringe 2, causing the locking thereof. The syringe 2 is removed by pushing again on the operating ring 136 to deactivate the tip 138a of the locking needle 138.

In the radiation-protection device 1 according to the invention, the shield 12 of transparent radiation-protection material can be made of lead glass. Preferably, it creates a magnifying glass effect to optimise vision of the end of the syringe plunger 8.

Generally, the outer surface 113 of the tubular radiation-protection structure 11 comprises at least a part of non-circular cross-section adapted to avoid or limit its rolling on a receiving surface.

1. A radiation-protection device (1) intended to be fitted onto a syringe (2) used for injecting a radioactive product, said syringe (2) comprising a cylindrical syringe body (3) having a front end (5) provided with a liquid suction and ejection hole (6) and a rear end (7) provided with an opening (9) into which is inserted a syringe plunger (8),
said radiation-protection device (1) comprising:
(i) a tubular radiation-protection structure (11), with a longitudinal axis (X), made at least partially of a radiation-protection material, said tubular radiation-protection structure (11) comprising—an inner surface (111) delimiting a central housing (112) adapted to receive at least part of said syringe body (3),—an outer surface (113),—a front opening (114) through which said front end (5) of said syringe body (3) is intended to emerge,—a rear opening (115) adapted for inserting and extracting said syringe body (3), and for operating said syringe plunger (8),—a side opening (116) that opens into said central housing (112), said side opening (116) comprising a rebate structure (117) and forming a side housing (118),
(ii) a shield (12) made of a transparent radiation-protection material, added into said side housing (118) and positioned in rest on said rebate structure (117), said radiation-protection device (1) also comprising locking means (14) to lock said shield (12) of transparent radiation-protection material in said side housing (118), wherein said locking means (14) comprise
a tenon structure (14a) arranged at one end (121) of said shield (12) of transparent radiation-protection material, adapted to cooperate with a complementary mortise structure (14b) arranged in said side housing (118), and
a retractable locking member (14c) adapted to cooperate with another end (122) of said shield (12) of transparent radiation-protection material, said locking member (14c) being able to be activated to lock said shield (12) of transparent radiation-protection material in position after introduction of said tenon structure (14a) into said mortise structure (14b), and to be deactivated to allow said shield (12) of radiation-protection material and said tubular radiation-protection structure (11) to be separated from each other.

2. The radiation-protection device (1) according to claim 1, wherein said end (122) of the shield (12) of transparent radiation-protection material cooperating with said locking member (14c) includes a shield rebate (19) for receiving said locking member (14c), said shield rebate (19) being delimited by a rebate bottom (191) and a rebate edge (192), a bottom face (20) of said locking member (14c) being intended to bear against said rebate bottom (191) of said shield rebate (19), to axially lock said shield (12) of radio-protective material, and a thickness of said locking member (14c) being intended to come between said rebate edge (192) of said shield rebate (19) and an end face (22) opposite said side housing (118), to longitudinally lock said shield (12) of radio-protective material.

3. The radiation-protection device (1) according to claim 1, wherein said side housing (118) is elongated in shape and comprises a major axis (A) that extends parallel to said longitudinal axis (X) of said tubular radiation-protection structure (11), said side housing (118) comprising an upstream end (118b) directed towards the rear opening (115) of said tubular radiation-protection structure (11) and a downstream end (118a) directed towards the front end (114) of said tubular radiation-protection structure (11),
wherein said mortise structure (14b) is arranged at said downstream end (118a) of the side housing (118),
wherein said tenon structure (14a) is arranged at one end (121) of said shield (12) of transparent radiation-protection material intended to be oriented towards said downstream end (118a) of the lateral housing (118),
and wherein said retractable locking member (14c) is intended to cooperate with the end (122) of said shield (12) of transparent radiation-protection material positioned on the side of the upstream end (118b) of said lateral housing (118).

4. The radiation-protection device (1) according to claim 1, wherein said retractable locking member (14c) consists of an elastic locking ring (14c) adapted to surround said tubular radiation-protection structure (11) and said associated shield (12) of transparent radiation-protection material.

5. The radiation-protection device (1) according to claim 4, wherein said retractable locking member (14c) in the form of an elastic locking ring is made of rubber.

6. The radiation-protection device (1) according to claim 4, wherein said retractable locking member (14c) in the form of an elastic locking ring comprises a flat structure (23) that projects from the outer surface (113) of said tubular radiation-protection structure (11), said projecting flat structure (23) being adapted to serve as a bearing structure, to be stably put on a receiving plane.

7. The radiation-protection device (1) according to claim 6, wherein said end (122) of the shield (12) of transparent radiation-protection material cooperating with said locking member (14c) includes a shield rebate (19) for receiving said locking member (14c), said shield rebate (19) being delimited by a rebate bottom (191) and a rebate edge (192), a bottom face (20) of said locking member (14c) being intended to bear against said rebate bottom (191) of said shield rebate (19), to axially lock said shield (12) of radio-protective material, and a thickness of said locking member (14c) being intended to come between said rebate edge (192) of said shield rebate (19) and an end face (22) opposite said side housing (118), to longitudinally lock said shield (12) of radio-protective material,
and wherein said flat structure (23) is diametrically opposed to said bottom face (20) of said locking member (14c).

8. The radiation-protection device (1) according to claim 4, wherein the outer surface (113) of said tubular radiation-protection structure (11) includes a slot (17) for the integration, at least partial, of said retractable locking member (14c) in the form of an elastic ring.

9. The radiation-protection device (1) according to claim 1, wherein the outer structure (113) of the tubular radiation-protection structure (11) comprises at least a part of non-circular cross-section adapted to avoid or limit its rolling on a receiving surface.
